(19) United States
(12) Patent Application Publication
(10) Pub. No.: US 2007/0014875 A1
(43) Pub. Date: Jan. 18, 2007

(54) Novel drug delivery system for proton pump inhibitors and process thereof

(76) Inventors: Mannalal Ramgopal Bajaj, Maharashtra (IN); Rajan Shantaram Samant, Maharashtra (IN); Bharat Babulal Shah, Maharashtra (IN)

Correspondence Address: BLANK ROME LLP, 600 NEW HAMPSHIRE AVENUE, N.W., WASHINGTON, DC, 20037, US

(21) Appl. No.: 10/574,666

(22) PCT Filed: Nov. 3, 2004
(86) PCT No.: PCT/IN04/00342

§ 371 Date: Apr. 4, 2006

(30) Foreign Application Priority Data
Nov. 5, 2003 (IN) 1164/MUM/2003

(51) Int. Cl. A61K 33/14

(52) U.S. Cl. 424/679; 424/680

(57) ABSTRACT

The present invention discloses a novel drug delivery system for proton pump inhibitors comprising benzimidazole compounds or their salts, preferably Rabeprazole or its salts and pharmaceutically acceptable excipients in powder form which is reconstitutable in a parenterally acceptable solvent to form an injectable solution.

### RELATED APPLICATION

This application claims priority from India National patent application serial No. 1164/MUM/2003, filed 5^{th} Nov. 2004.

### TECHNICAL FIELD OF THE INVENTION

This invention relates to a novel drug delivery system for proton pump inhibitors (PPIs). More particularly, this invention relates to a stable, pharmaceutically acceptable, lyophilized injectable form of Rabeprazole. This invention further relates to a process for preparation of the said Lyophilized injectable form.

### BACKGROUND AND PRIOR ART

Proton pump inhibitors (PPIs) form the emerging anti-ulcer compounds and have already overtaken H2 antagonists like Ranitidine. PPIs are now the drugs of choice for stomach and duodenal ulcers. They are also effectively used to relieve symptoms of esophagitis and acute gastro-esophageal efflux. PPIs are also used to alleviate Helicobacter pylori infection which is considered to be the root cause of stomach ulcers. PPI's block the production of stomach acids by inhibiting a system in the stomach known as proton pump, also referred to as hydrogen-potassium adenosine triphosphate enzyme system.

Omeprazole (also esomeprazole), Lansoprazole, Pantoprazole and Rabeprazole are the leading commonly used proton pump inhibitors (PPIs). Owing to the close similarity between these PPIs, the formulations and dosage forms can be similarly formulated for the entire group of compounds based on a process developed for any one of the group of PPIs.

Rabeprazole (marketed as Aciphex in USA and other countries) is available only in tablet form or as delayed release tablets in NDDS.

Rabeprazole is currently administered by employing any suitable route of administration such as rectal, transdermal and like forms with effective dosage of active ingredient; however oral administration has hitherto been the preferred route. Reported oral dosage forms are tablets, troches, dispersions, suspensions, solutions, capsules and the like.

International Patent application WO9601624 describes a pharmaceutical formulation in the dosage form of multiple unit tablets which contains active ingredient, an acid labile H+K+ATPase inhibitor like Rabeprazole, or alkaline salts thereof.

Oral dosage forms for Rabeprazole are also disclosed in U.S. Pat. No. 5,035,899 and International Patent application WO97/12580 and WO97/25030.

Compositions of Rabeprazole suitable for rectal administration are described in European Patent 645140.

Further, the injections for PPI's have recently been developed. Japanese Patent unexamined Publication no. JP167587/1984 describes the process for preparation of injection of Omeprazole. The process comprises dissolving sodium salt of Omeprazole in sterilized water, filtering and lyophilizing the solution to give lyophilized product. This lyophilized product is dissolved in a mixture of polyethylene glycol 400 for injection, sodium dihydrogen phosphate and sterilized water.

For Lansoprazole the lyophilized injection is prepared by dissolving lyophilized product of Lansoprazole in a mixture of acid and at least one of ethanol, propylene glycol and polyethylene glycol as described in Japanese unexamined patent no. JP138213/1990.

Freeze dried injectable formulation of Pantoprazole is described in International Patent application WO0241919. Lyophilization of the aqueous solutions of Pantoprazole, ethylenediamine tetra acetic acid and/or a suitable salt thereof, and sodium hydroxide and/or sodium carbonate is disclosed.

Freeze dried formulations for Omeprazole and Lansoprazole, as described in International Patent application WO9402141, comprise the benzimidazole compounds or their salts to which is added an aqueous solvent wherein the pH is not less than 9.5 and not more than 11.5.

However, there is no formulation or delivery system for Rabeprazole, in particular, in injectable form. We have developed lyophilized, stable injectable dosage form of Rabeprazole, the process of which could also be applied for other PPIs like, Omeprazole, Lansoprazole, Pantoprazole, etc.

### OBJECTIVE OF THE INVENTION

The objective of the present invention is to provide a stabilized pharmaceutically acceptable dosage form of proton pump inhibitors and in particular stabilized lyophilized (freeze dried) injection of Rabeprazole.

### SUMMARY OF THE INVENTION

The present invention provides a pharmaceutical composition comprising in powder form, (a) Rabeprazole or its salts in a therapeutically effective total amount constituting about 8% to about 77% by weight and (b) mannitol 19% to 88%, by weight, of the composition.

Particularly the said composition is reconstitutable in a parenterally acceptable solvent liquid, preferably an aqueous liquid, to form an injectable solution.

The said composition is prepared by a process which comprises lyophilization of an aqueous solution comprising Rabeprazole, mannitol, alkaline compounds and optionally other excipients to form a readily reconstitutable powder.

The present invention provides a novel drug delivery system for proton pump inhibitors which comprises, reconstituting a unit dosage of the composition in a physiologically acceptable volume of a parenterally acceptable solvent liquid, to form an injectable solution.

The said salts of Rabeprazole may be in the form of alkaline metal salts or alkaline earth metal salts. The said alkaline metal salts may be sodium or potassium and the said alkaline earth metal salts may be calcium or magnesium.

The said system comprises Rabeprazole sodium, mannitol and alkaline compounds in the form of a stabilized lyophilized injection. The said alkaline compound is preferably sodium hydroxide. The pH of the said system after reconstitution is between 9-11.

Further the present invention provides a process for preparation of the said drug delivery system which comprises dissolving sodium hydroxide in Water for injection to adjust the pH above 12.0, adding Mannitol and Rabeprazole sodium to the above said solution; maintaining the pH the same; making up the volume with water for injection; filtering the said solution aseptically through 0.22μ filter paper; and filling the said filtered solution in previously sterilized 10 ml vial, after partial bunging; loading the vials into a lyophilizer and lyophilizing the said solution to obtain the said drug delivery system.

The process of stabilization and pharmaceutical excipients or ingredients used therein provides unique and novel stability and efficacy to the composition.

The term, “Rabeprazole” is not intended to be limited only to Rabeprazole, but is intended to include all benzimidazole compounds and their pharmaceutically acceptable salts.

### DETAILED DESCRIPTION

The present invention provides a pharmaceutical composition comprising in powder form, (a) Rabeprazole or its salts in a therapeutically effective total amount constituting about 8% to about 77% by weight and (b) mannitol 19% to 88%, by weight, of the composition.

Particularly the said composition is reconstitutable in a parenterally acceptable solvent, preferably an aqueous liquid, to form an injectable solution.

Stability and efficacy are incorporated into the composition by the novel process and pharmaceutical excipients and ingredients employed.

The said composition is prepared by a process which comprises lyophilization of an aqueous solution containing Rabeprazole, mannitol, alkaline compounds and optionally other excipients to form a readily reconstitutable powder.

The present invention provides a novel drug delivery system for proton pump inhibitors which comprises, reconstituting a unit dosage of the composition in a physiologically acceptable volume of a parenterally acceptable solvent liquid to form an injectable solution.

The said salts of Rabeprazole may be in the form of alkaline metal salts or alkaline earth metal salts. The said alkaline metal salts may be sodium or potassium and the said alkaline earth metal salts may be calcium or magnesium.

The said system comprises Rabeprazole sodium, mannitol and alkaline compounds in the form of stabilized lyophilized injection.

The said alkaline compound is preferably sodium hydroxide. The pH of the said system after reconstitution is between 9-11.

Further the present invention provides a process for preparation of the said drug delivery system comprising dissolving sodium hydroxide in Water for injection to adjust the pH above 12.0, adding Mannitol and Rabeprazole sodium to the said above solution; maintaining the pH the same; making up the volume with water for injection; filtering the said solution aseptically through 0.22μ filter paper and filling the said filtered solution in previously sterilized 10 ml vials; the solution temperature should be maintained at 10° C.±2° C. throughout the procedure, after partial bunging, loading the vials into a lyophilizer and lyophilizing the said solution to obtain the said drug delivery system.

Rabeprazole disclosed hereinabove is present in a reconstitutable powder composition in a total amount of about 8% to about 77%, preferably about 19% to about 62%.

The said Mannitol is in the range of 19% to 88%, preferably 30-80%.

Benzimidazole compounds and/or their salts are stable in the alkaline pH range and their stability decreases with lowering pH values. Hence, the pH of the composition upon reconstitution should be about 9-11.

The present invention will now be further illustrated by the following non-limiting example.

### EXAMPLE

Sodium hydroxide was dissolved in Water for Injection (approx-38 liters) to make 0.01M solution. The pH of the said solution was adjusted above 12.0. Mannitol (600 gm) and Rabeprazole sodium (447 gm) were added to the above solution, maintaining the pH and making up the volume to 40 liters. The above solution was filtered aseptically through 0.22μ filter paper and 2.0 ml of filtered solution was filled in previously sterilized 10 ml vials, the solution temperature should be maintained at 10° C.±2° C. throughout the procedure. After partial bunging, the vials were loaded into the lyophilizer. The lyophilizer shelf temperature was maintained at 5° C.±2° C. during the charging operation.

After the vial loading, the vials were chilled to −40° C. and held at this temperature for 2 hours. At the end of 2 hours, the condenser was chilled to below −40° C. Next, the condenser was vaccumized to less than 200 micron before opening the butterfly valve. After opening the butterfly valve, during first hour, the lyophiliser chamber conditions were allowed to stabilize. Next 4 hours, heating medium temperature was maintained at −20° C. Next 6 hours, the heating medium temperature was maintained at −10° C. Next 4 hours the heating medium temperature was maintained at −5° C.

Next 4 hours, the heating medium temperature maintained at 0° C. Next 1 hour heating medium temperature maintained at 5° C. Next 2 hours heating medium temperature maintained at 10° C. Next 1 hour heating medium temperature maintained at 15° C. Next 1 hour heating medium temperature maintained at 20° C. Next, the heating medium temperature was maintained at 30° C. until 4 micron point was reached.

The cycle was terminated after reaching 4 micron end point, butterfly valve was closed and break the vacuum, with sterile filtered Nitrogen. After the batch completion, full stoppering and sealing was carried out.

The resulting formulation contained the following components in the following amounts.

Rabeprazole Sodium for injection 20 mg/vial

| | | Qty per | | Qty per 300 ml | | Qty per | |
|---|---|---|---|---|---|---|---|
| Ingredients | Specification | vial | | (150 vials) | | 20,000 vials | |
| Rabeprazole | IH | 20 | mg | 3.352 | gm | 447 | gm |
| Sodium eq | | | | | | | |
| to Rabeprazole | | | | | | | |
| Mannitol | IP | 30 | mg | 4.5 | gm | 600 | gm |
| Sodium | IP | 0.8 mg to | | 120 | mg | 16 | gm |
| hydroxide | | adjust pH | | | | | |
| Water for | IP | 2.0 | ml | 300 | ml | 40 | lts |
| Injection before | | | | | | | |
| lyophilization | | | | | | | |
| qs. | | | | | | | |

The example mentioned above for Rabeprazole and the process for lyophilization, the conditions including pH and pharmaceutically acceptable inactive ingredients used therein are also applicable for other Proton Pump Inhibitors and lyophilized dosage forms thereof.

Stability studies were carried out as per ICH guidelines for the said Rabeprazole composition at accelerated conditions and the results obtained were satisfactory.

While the present invention is described above in connection with preferred or illustrative embodiments, these embodiments are not intended to be exhaustive or limiting of the invention. Rather, the invention is intended to cover all alternatives, modifications and equivalents included within its spirit and scope, as defined by the appended claims.

1. A stabilized drug delivery system for proton pump inhibitors comprising a benzimidazole compound or a salt thereof and a pharmaceutically acceptable excipient in powder form, which is reconstitutable in a parenterally acceptable solvent to form an injectable solution.

2. The drug delivery system as claimed in claim 1, wherein said salt is in the form of alkaline metal salt or alkaline earth metal salt.

3. The drug delivery system as claimed in claim 2, wherein said alkaline metal salts is sodium or potassium.

4. The drug delivery system as claimed in claim 2, wherein said alkaline earth metal salts is calcium or magnesium.

5. The drug delivery system as claimed in claim 1, wherein said system comprises Rabeprazole sodium, mannitol, alkaline compounds and water for injection.

6. The drug delivery system as claimed in claim 1, wherein said system is in the form of a stabilized lyophilized injection.

7. The drug delivery system as claimed in claim 5, wherein said alkaline compound is sodium hydroxide.

8. The drug delivery system as claimed in claim 1, wherein the pH of said system is between 9-11.

9. The drug delivery system as claimed in claim 1, wherein the said benzimidazole compound is present in the range of 8% to 77% by weight of the total composition.

10. The drug delivery system as claimed in claim 1, wherein the said excipient is present in the range of 19% to 88% by weight of the total composition.

11. (canceled)

12. The drug delivery system for proton pump inhibitor prepared by process as claimed in claim 18.

13-14. (canceled)

15. The drug delivery system as claimed in claim 1, wherein the benzimidazole compound or the salt thereof is Rabeprazole or its salt.

16. The drug delivery system as claimed in claim 9, wherein the said benzimidazole compound is present in the range of 19-62% by weight of the total composition.

17. The drug delivery system as claimed in claim 10, wherein the said excipient is present in the range of 30%-88% by weight of the total composition.

18. A process for preparation of the said drug delivery system comprising the steps of
(a) dissolving sodium hydroxide in Water For Injection to adjust the pH above 12.0 to form a solution;
(b) adding Mannitol and Rabeprazole sodium to said solution while maintaining the pH of the said solution;
(c) making up the volume with water for injection;
(d) filtering the said solution aseptically through 0.22μ filter paper;
(e) filling the said filtered solution in previously sterilized 10 ml vials;
(f) maintaining the temperature of the injectable solution at 10° C.±2° C. throughout the process;
(g) loading the vials into lyophilizer after partial lounging; and
(f) lyophilizing the solution to obtain a powder form the drug delivery system which is reconstitutable in a parenterally acceptable solvent to form an injectable solution.
